(19) United States
(12) Patent Application Publication
(10) Pub. No.: US 2018/0110569 A1
(43) Pub. Date: Apr. 26, 2018

(54) SURGICAL INSTRUMENT WITH LED LIGHTING AND ABSOLUTE ORIENTATION

(71) Applicant: Prichard Medical, LLC, Rocky River, OH (US)

(72) Inventor: Joseph Prichard Drain, Cleveland Heights, OH (US)

(21) Appl. No.: 15/619,747

(22) Filed: Jun. 12, 2017

Related U.S. Application Data
(60) Provisional application No. 62/413,355, filed on Oct. 26, 2016.

(51) Int. Cl. A61B 34/20; A61B 90/30

(52) U.S. Cl. CPC A61B 34/20; A61B 90/35; A61B 90/30

(57) ABSTRACT

The present disclosure relates generally to surgical instruments. More particularly, this disclosure relates to surgical instruments having high intensity lighting and absolute orientation sensors. The instruments of the present disclosure may be used in any suitable procedure or treatment which would benefit from high intensity lighting of the area to be treated or of interest, as well as knowing the orientation of such instrument in three-dimensional space.

### CROSS REFERENCE TO RELATED APPLICATION

This application claims the benefit of U.S. Provisional Application No. 62/413,355 filed on Oct. 26, 2016, the contents of which are incorporated herein by this reference as though set forth in their entirety.

### FIELD OF USE

The disclosure relates generally to surgical and medical instruments. More particularly, this disclosure relates to surgical and medical instruments having high intensity lighting and absolute orientation sensors. The instruments of the present disclosure may be used in any suitable procedure or treatment which would benefit from high intensity lighting of the area to be treated or of interest, as well as knowing the orientation of such instrument in three-dimensional space. While reference is made herein to surgical instruments in particular, it should be understood that this disclosure is directed to medical, dental, or other instruments used in the treatment of humans or animals requiring lighting and absolute orientation knowledge.

### BACKGROUND

Adequate lighting of an area to be treated is of great importance for any surgical or medical procedure. Operating room lighting may allow a surgeon to have an improved view of the surgical field. In this regard, lighting arrays may be provided in operating rooms or outpatient clinics to assist in illuminating the surgical field. Alternatively, bulky fiber-optic cable can also be used to illuminate the surgical field by being placed in or near the surgical field. Additional light emitters, such as headlamps and the like, worn by medical personnel may also be employed. However, in all these instances the light emitter is generally provided some distance away from the surgical instrument, and/or away from the surgical field, and/or in an orientation not conducive to illuminating critical portions of the surgical instrument or the surgical area. As such, the surgical instrument and light emitters are separated and shadows may be cast on the surgical field by the user, the electrosurgical instrument, or other obstructions (e.g., table drapes, other devices utilized during surgery, user's hand, etc.).

In addition, determining the optimal or desired alignment and/or orientation of surgical or medical instruments, such as for the proper placement of implants into the body, and maintaining such alignment and/or orientation as required for the selected procedure has long been a challenge. The surgeon must be provided with efficient and reliable feedback as to the orientation of the instrument in three-dimensional space with respect to the patient's body or treatment area in order to quickly and accurately correct any errors or issues with the orientation. Failure to do so may cause damage to the patient. Orientation data can be exploited in multiple settings, several of which occur in the operating room: hand-held, powered, or robotic surgical instruments all stand to benefit from real-time calculation of their precise orientation, location, as well as their linear and angular acceleration and velocity.

As example of this application that take can take place inside or outside of the operating room, doctors regularly perform a biopsy procedure involving the insertion of a biopsy needle into an object to be examined (e.g., a living body such as a human body) and extracting a tissue sample from a biopsy region in the object. Generally, in order to reduce the physical burden on patient, it is desirable to insert the biopsy needle near the biopsy region to reliably and accurately extract a tissue sample from the biopsy region. Accordingly, it has been customary in a biopsy to carry out a stereoscopic image capturing process in which radiation is applied to an object to be examined, and a stereoscopic image of the object is acquired, and then to calculate a three-dimensional position of the biopsy region from the stereoscopic image, before the biopsy procedure begins.

However, even if the three-dimensional position of the biopsy region is accurately mapped in advance of the procedure, it is possible that the needle may deviate from the desired puncture path (e.g., an interference with a blood vessel in the object). As a result, the inserted biopsy needle does not follow the desired puncture path, and then the positional relationship, i.e., distance and direction relationship, between the biopsy region from which a tissue sample is to be extracted and the opening of the biopsy needle. To solve the problem, there have been proposed various technologies for aspirating and sampling an appropriate amount of tissue from a biopsy region through a biopsy needle without the need for inserting the biopsy needle again. This is just another manner in which this technology can be deployed in the medical setting.

There is a need for surgical and medical instruments to include a high intensity light source and/or absolute orientation sensors to improve the effectiveness of such instruments.

### SUMMARY

The following presents a simplified overview of the example embodiments in order to provide a basic understanding of some aspects of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented later.

In accordance with embodiments herein, the present disclosure is directed to surgical and medical instruments and devices having absolute orientation sensors and/or high intensity lighting. The instruments of the present disclosure may be used in any suitable procedure or treatment which would benefit from knowing the orientation of the surgical instrument or high intensity lighting of the area to be treated or of interest. While reference is made herein to surgical instruments in particular, it should be understood that this disclosure is directed to medical, dental, or other instruments used in the treatment of humans or animals requiring lighting and absolute orientation. Absolute orientation means that the absolute orientation sensors may not require calibration against a known point or plane in order to provide orientation related information. The absolute orientation sensor may comprise an accelerometer, gyroscope, and magnetometer, and may be able to generate an absolute orientation by using the Earth itself as a reference point or plane, by sensing the Earth's magnetic field, and by extension, the Earth's magnetic core, rather than an arbitrarily determined point or plane.

One embodiment may be a surgical instrument comprising: a device processor operable for controlling one or more components of the surgical instrument; an absolute orientation sensing component operatively coupled to the device processor and controlled in part by the device processor, wherein the absolute orientation sensing component may comprise an accelerometer, gyroscope, and magnetometer, wherein the absolute orientation sensing component may be operable to detect a plurality of orientation data associated with at least one orientation condition of the surgical instrument relative to the Earth's magnetic field without requiring calibration by a separate arbitrary reference plane, and generate a plurality of orientation status data on at least a portion of the plurality of detected orientation data; and at least one power source operatively coupled to the absolute orientation sensing component, wherein the at least one power source may be operable to generate a supply of power for operation of the device processor and the absolute orientation sensing component. The surgical instrument may further comprising a display operatively coupled to the device processor, wherein the display may be operable to display at least a portion of the plurality of generated orientation status data thereon. The plurality of orientation data may comprise at least one from the group consisting of a location data, pitch data, roll data, and yaw data. The location data may comprise x, y, and z coordinate values. The plurality of orientation data may further comprise at least one selected from the group consisting of angular velocity data, acceleration data, magnetic field strength data, linear acceleration data, linear velocity data, gravity data, and temperature data. The surgical instrument may further comprise an input/output device operably coupled to the device processor and controlled in part by the device processor; wherein the input/out device may be operable to transmit at least a portion of the plurality of generated orientation status data to a remote device. The surgical instrument may further comprise an audio component operatively coupled to the device processor and controlled in part by the device processor, wherein the device processor may be further operable to generate at least one audio response based in response to at least a portion of the plurality of generated orientation status data for output by the audio component. The surgical instrument may further comprise at least one light source operatively coupled to the device processor and controlled in part by the device processor, wherein the at least one light source may be located on the surgical instrument such that the at least one light source may be operable to direct light towards a specified area on an associated patient. The at least one light source may be a high lumen light source. The at least one light source may be a light emitting diode, compact fluorescent, incandescent, fluorescent, or halogen bulb. The device processor may be operable to activate the at least one light source when the surgical device is in use. The surgical instrument may be drills, drivers, saws, wire insertion devices, burr, awls, scalpels, suction, retraction devices, mallets, biopsy needles, unpowered drills, unpowered drivers, unpowered saws, unpowered wire inserters, unpowered burrs, and the like. Additionally, the surgical instrument may be a robotic surgical device, such as robotic arms or other assisted medical devices which provide a user or robot with real-time orientation data.

Another embodiment may be a surgical instrument comprising a device processor operable for controlling one or more components of the surgical instrument; an absolute orientation sensing component operatively coupled to the device processor and controlled in part by the device processor, wherein the absolute orientation sensing component may comprise an accelerometer, gyroscope, and magnetometer, wherein the absolute orientation sensing component may be operable to detect a plurality of orientation data associated with at least one orientation condition of the surgical instrument relative to the Earth's magnetic field without requiring calibration by a separate arbitrary reference plane, and generate a plurality of orientation status data on at least a portion of the plurality of detected orientation data; at least one power source operatively coupled to the absolute orientation sensing component, wherein the at least one power source may be operable to generate a supply of power for operation of the device processor and the absolute orientation sensing component; and an audio component operatively coupled to the device processor and controlled in part by the device processor, wherein the device processor may be further operable to generate at least one audio response based in response to at least a portion of the plurality of generated orientation status data for output by the audio component; wherein the plurality of orientation data may comprise at least one from the group consisting of a location data, pitch data, roll data, and yaw data, angular velocity data, acceleration data, magnetic field strength data, linear acceleration data, linear velocity data, gravity data, and temperature data; and wherein the location data may comprise x, y, and z coordinate values. The surgical instrument may further comprise an input/output device operably coupled to the device processor and controlled in part by the device processor; wherein the input/out device may be operable to transmit at least a portion of the plurality of generated orientation status data to a remote device. The surgical instrument may further comprise a display operatively coupled to the device processor, wherein the display may be operable to display at least a portion of the plurality of generated orientation status data thereon. The surgical instrument may further comprise at least one light source operatively coupled to the device processor and controlled in part by the device processor, wherein the at least one light source may be located on the surgical instrument such that the at least one light source may be operable to direct light towards a specified area on an associated patient; wherein the light source may be a high lumen light source; wherein the light source may be a light emitting diode, compact fluorescent, incandescent, fluorescent, or halogen bulb; wherein the device processor may be operable to activate the light source when the surgical device may be in use.

Another embodiment of the surgical instrument may comprise: a device processor operable for controlling one or more components of the surgical instrument; at least one light source operatively coupled to the device processor and controlled in part by the device processor, wherein the at least one light source may be located on the surgical instrument such that the at least one light source may be operable to direct light towards a specified area on an associated patient; wherein the light source may be a high lumen light source; wherein the light source may be a light emitting diode, compact fluorescent, incandescent, fluorescent, or halogen bulb; wherein the device processor may be operable to activate the light source when the surgical device may be in use; and at least one power source operatively coupled to the light source, wherein the at least one power source may be operable to generate a supply of power for operation of the device processor and the light source. The surgical instrument may further comprise an absolute orientation sensing component operatively coupled to the device processor and controlled in part by the device processor, wherein the absolute orientation sensing component may comprise an accelerometer, gyroscope, and magnetometer, wherein the absolute orientation sensing component may be operable to detect a plurality of orientation data associated with at least one orientation condition of the surgical instrument relative to the Earth's magnetic field without requiring calibration by a separate arbitrary reference plane, and generate a plurality of orientation status data on at least a portion of the plurality of detected orientation data; and further comprising an audio component operatively coupled to the device processor and controlled in part by the device processor, wherein the device processor may be further operable to generate at least one audio response based in response to at least a portion of the plurality of generated orientation status data for output by the audio component; wherein the plurality of orientation data may comprise at least one from the group consisting of a location data, pitch data, roll data, and yaw data, angular velocity data, angular acceleration data, magnetic field strength data, linear velocity and linear acceleration data, gravity data, and temperature data; and wherein the location data may comprise x, y, and z coordinate values. The surgical instrument may further comprise an input/output device operably coupled to the device processor and controlled in part by the device processor; wherein the input/out device may be operable to transmit at least a portion of the plurality of generated orientation status data to a remote device. The surgical instrument may further comprise a display operatively coupled to the device processor, wherein the display may be operable to display at least a portion of the plurality of generated orientation status data thereon.

Still other advantages, aspects and features of the subject disclosure will become readily apparent to those skilled in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious aspects all without departing from the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is a block diagram of a surgical instrument.

FIG. 2 is a perspective illustration of one embodiment of a surgical instrument that is a surgical awl according to the present disclosure.

FIG. 3 is a side plan illustration of one embodiment of a surgical instrument that is a surgical awl according to the present disclosure.

FIG. 4 is a front perspective illustration of an alternative embodiment of a surgical instrument that is a surgical awl according to the present disclosure.

FIG. 5 is a rear perspective illustration of an alternative embodiment of a surgical instrument that is a surgical awl according to the present disclosure.

FIG. 6 is a side perspective illustration of an embodiment of a surgical instrument that is a surgical drill according to the present disclosure.

FIG. 7 is a front perspective illustration of an embodiment of a surgical instrument that is a surgical drill according to the present disclosure.

FIG. 8 is an illustration of one embodiment of a surgical instrument that is a biopsy needle according to the present disclosure.

FIG. 9 is an illustration of one embodiment of an absolute orientation sensor and display.

FIG. 10 is an illustration of one embodiment of a surgical instrument that is a robotic arm according to the present disclosure.

### DETAILED DESCRIPTION OF THE ILLUSTRATIVE EMBODIMENTS

This description provides examples not intended to limit the scope of the appended claims. The figures generally indicate the features of the examples, where it is understood and appreciated that like reference numerals are used to refer to like elements. Reference in the specification to “one embodiment” or “an embodiment” or “an example embodiment” means that a particular feature, structure, or characteristic described is included in at least one embodiment described herein and does not imply that the feature, structure, or characteristic is present in all embodiments described herein.

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware embodiments. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, may be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that may direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, may be implemented by special purpose hardware-based computer systems that perform the specified functions or steps, or combinations of special purpose hardware and computer instructions.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

The absolute orientation sensor may comprise an accelerometer, gyroscope, and magnetometer, and may be able to generate an absolute orientation by using the Earth itself as a reference point or plane, by sensing the Earth's magnetic field, and by extension, the Earth's magnetic core, rather than an arbitrarily determined point or plane. When the absolute orientation sensor is connected to a surgical instrument the orientation of the absolute orientation sensor may be extrapolated to determine the orientation of the surgical instrument. This orientation of the surgical instrument may be conveyed to a user through display on a digital display that is located on the surgical instrument, or the orientation information may be transmitted to an external electronic device. The absolute orientation sensor may be attachable to substantially any surgical device. Additionally, the surgical instrument may have a high lumen light source to assist a user in viewing a place of a body to be operated upon.

The accompanying drawings incorporated herein and forming a part of the specification illustrate the example embodiments.

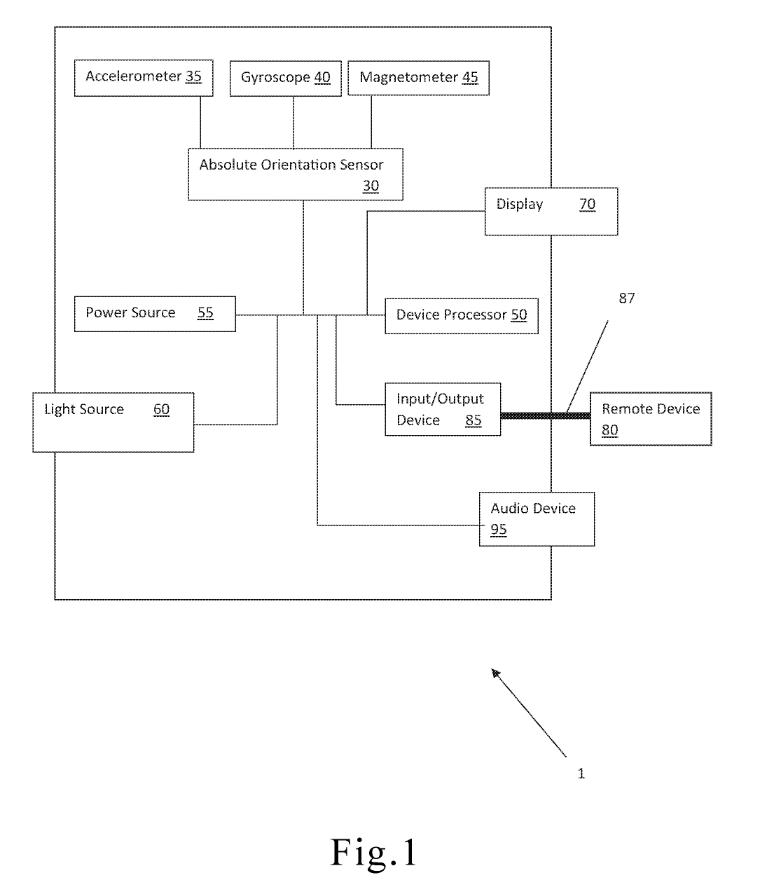

FIG. 1 is a block diagram of a surgical instrument. As shown in FIG. 0, the surgical instrument 1 may comprise an absolute orientation sensor 30, accelerometer 35, gyroscope 40, magnetometer 45, device processor 50, power source 55, light source 60, display 70, input/output device 85, remote device 80, communication link 87, and audio device 95.

The accelerometer 35, gyroscope 40, and magnetometer 45 may be components of the absolute orientation sensor 30. An accelerometer measures acceleration, such that an accelerometer at rest on the surface of the Earth measures a positive acceleration of 9.81 m/s, and an accelerometer in free fall towards the center of the Earth measures an acceleration of 0 m/s. A gyroscope, historically is a spinning wheel or disc in which the axis of rotation is free to assume any orientation by itself, but in this instance a microelectrical mechanical system, is useful for measuring or maintaining orientation, providing information about angular acceleration, velocity, and position. A magnetometer is an instrument that measures magnetism—either magnetization of magnetic material like a ferromagnet, or the direction, strength, or the relative change of a magnetic field at a particular location. The combination of an accelerometer, gyroscope and magnetometer may comprise an Inertial Measurement Unit and are often included in inertial navigation systems.

The power source 55 may provide power to the absolute orientation sensor 30, device processor 50, light source 60, display 70, input/output device 85, and audio device 95. The device processor 50 may be operatively coupled to, and control, the absolute orientation sensor 30, device processor 50, light source 60, display 70, input/output device 85, and audio device 95.

The device processor 50 may be, or may comprise, any suitable microprocessor or microcontroller, for example, a low-power application-specific controller (ASIC) and/or a field programmable gate array (FPGA) designed or programmed specifically for the task of controlling a device as described herein, or a general purpose central processing unit (CPU), for example, one based on 80×86 architecture as designed by Intel™ or AMD™, or a system-on-a-chip as designed by ARM™. The device processor 50 may be coupled (e.g., communicatively, operatively, etc.) to auxiliary devices or modules of the surgical instrument 1 using a bus or other coupling.

The absolute orientation sensor 30 may capture, in real-time, various orientation variables of the surgical instrument 1, including location, pitch, roll, yaw angular acceleration, velocity, and position; linear acceleration, velocity, and position, as well as magnetic field strength, linear acceleration, gravity, and/or temperature. Location may include x, y, and z coordinate values, and other indicators of position in three dimensional space.

The absolute orientation sensor 30 may be configured to generate orientation information that is particular to the tool being used. For example, when the absolute orientation sensor 30 is on the surgical instrument 1, particularly important orientation information may include information relating to location, pitch, roll, and yaw of the tip or active portion of the surgical instrument 1, such as a drill portion of an awl, or other manual, powered, hand held, robotic or other surgical device or tool. The absolute orientation sensor 30 may also be calibrated such that the orientation information generated by the absolute orientation sensor 30 is directly readable on the tip or active portion of the surgical instrument 1 by calculating the differences in orientation of the absolute orientation sensor 30 and tip or active portion of the surgical instrument 1 during a calibration step. Importantly, while it may be recommended to calibrate the absolute orientation sensor 30 relative to the tip or active portion of the surgical instrument 1, it is not required that the absolute orientation sensor 30 be calibrated against an arbitrary point or plane. The combination of accelerometer 35, gyroscope 40, and magnetometer 45 allow the absolute orientation sensor 30 to self-calibrate by using the Earth's magnetic field and/or the Earth's core as reference points or planes. In one embodiment, the absolute orientation sensor 30 may comprise a microelectricalmechanical system (MEMS) multi-axis gyroscope, multi-axis accelerometer, and a multiaxial geomagnetic sensor that is able to generate output information comprising quaternion, Euler angles, rotation vector, linear acceleration, gravity, and heading. A combination of multiaxis gyroscope, multiaxis accelerometers, and multiaxis geomagnetic sensors (magnetometer) may comprise an Inertial Measurement Unit mode.

In one embodiment, the absolute orientation sensor 30 may comprise an accelerometer 35 that outputs absolute orientation in the “X”, “Y”, and “Z” axis as well as angular velocity, acceleration, magnetic field strength, linear acceleration, gravity, and temperature. “X” and “Y” orientation of the instrument may output onto a display, providing the user an absolute value of where the surgical instrument is held in space. The addition of the gyroscope 40 may allow the absolute orientation sensor 30 to measure tilt, rotation, and changes in angular momentum. The addition of the magnetometer 45 allows for the measurement of the magnetic field of the Earth and the Earth's core. The combination of the accelerometer 35, gyroscope 40, and magnetometer 45 allows the absolute orientation sensor 30 to calibrate itself against a fixed point, the Earth's core, and thereby be accurate regardless of where the absolute orientation sensor 30 is in use.

This may allow the user to move the surgical instrument and receive real time quantified feedback about the angle and orientation of the surgical instrument. This may aid in surgical instrument alignment and its use, in one instance the placement of screws. The absolute orientation sensor 30 comprise any suitable sensors and related components for determining orientation as is known in the art. Examples include, but are not limited to, accelerometers 35, gyroscopes 40, magnetometers 45, and the like, and combinations thereof. The absolute orientation sensor 30 may interface wirelessly or wired, or both, with the surgical instrument or components thereof to control its operation. The orientation sensor 30 may be powered by any suitable means, including, but not limited to, connection to the power source of the surgical instrument, battery, rechargeable battery, connection to a separate power source, and the like. The surgical instrument 1 may also comprise power supply. The power supply may comprise one or more batteries and/or other power storage device (e.g., capacitor) and/or a port for connecting to an external power supply. The one or more batteries may be rechargeable. The one or more batteries may comprise a lithium-ion battery (including thin film lithium ion batteries), a lithium-ion polymer battery, a nickel—cadmium battery, a nickel metal hydride battery, a lead—acid battery, combinations thereof, and the like. For example, an external power supply may supply power to the surgical instrument 1 and a battery may store at least a portion of the supplied power.

Accordingly, while a doctor generally has the use of his or her eyes when performing a medical procedure, it may be difficult to be able to see or otherwise discern the exact orientation of a particular surgical instrument, and use of the absolute orientation sensor 30 may be able to provide doctors with the exact orientation of a particular surgical instrument. It may be especially difficult to determine the orientation of this surgical instrument when part of the surgical instrument is inside a patient.

The absolute orientation sensor 30, utilizing its components, may determine various orientation factors of the surgical instrument 1, which may include location data, pitch data, roll data, and yaw data, angular velocity data, acceleration data, magnetic field strength data, linear acceleration data, gravity data, and temperature data. Location data may comprise x, y, and z coordinate values. Once orientation factors are determined or generated by the absolute orientation sensor 30, the absolute orientation sensor 30 may transmit a signal to the device processor 50. The device processor 50 may then transmit the signal in one or more ways to one or more different locations.

In one embodiment, the device processor 50 may transmit the signal comprising the orientation information to the display 70. The display 70 may digitally display the information received such that the user may read the information off of the display 70. In another embodiment, the device processor 50, may transmit the signal comprising the orientation information to the input/output device 85, which may then communicate via a communication link 87 with the remote device 80. The communication link 87 may be a wireless signal, wired signal, or other communication protocol. The remote device 80 may then display, or otherwise use, the orientation information. In another embodiment, the device processor 50 may transmit a signal, based on the received orientation information, to the audio device 95 to generate a sound. For example, where the orientation information received by the device processor 50 indicates that the surgical instrument 1 is not within specified parameters, the device processor 50 may send a signal to the audio device 95 to generate a sound, which signals to a user that the surgical instrument 1 should have its orientation corrected. The degree or intensity of the sound may correspond to how far outside the specified parameters the surgical instrument 1 is.

In an alternative embodiment, the device processor 50 may send a signal to the light source 60 controlling the light source 60 when one or more other conditions are met. For example, the device processor 50 may activate, or deactivate, the light source 60 when the surgical instrument 1 is in use, when one or more orientation conditions are met, when one or more orientation conditions are not met, or another trigger event. The light source 60 may include compact fluorescence lights, light emitting diodes, incandescent, fluorescent, halogen, and other types of lights.

In one embodiment of the surgical instrument 1, the information received by the remote device 80 may be used to create a real-time virtual image or representation of the surgical instrument 1 by the remote device 80.

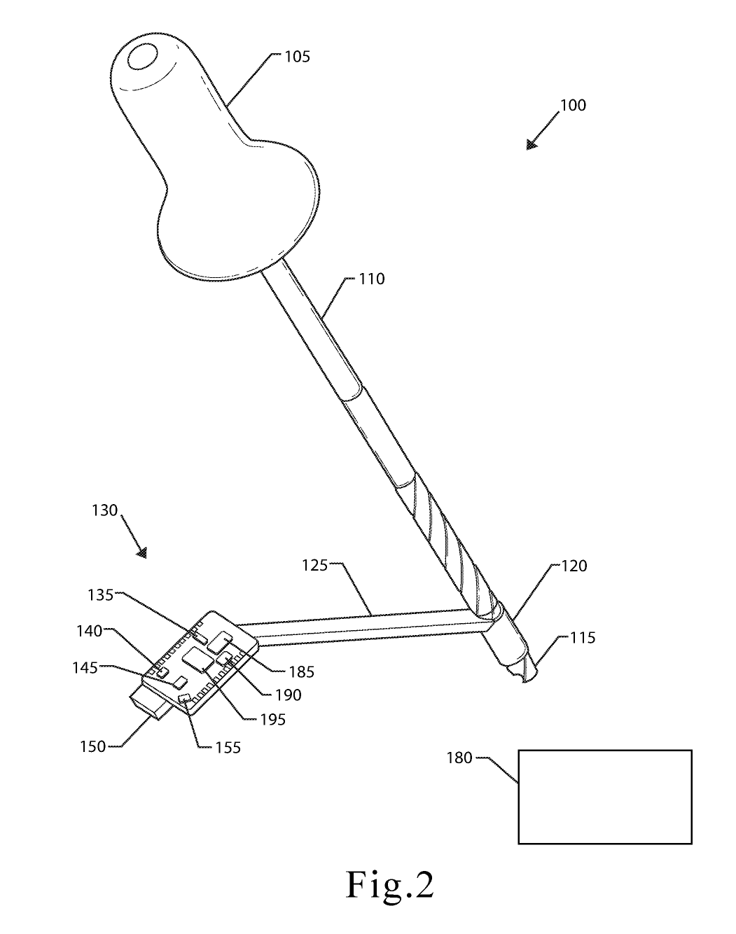

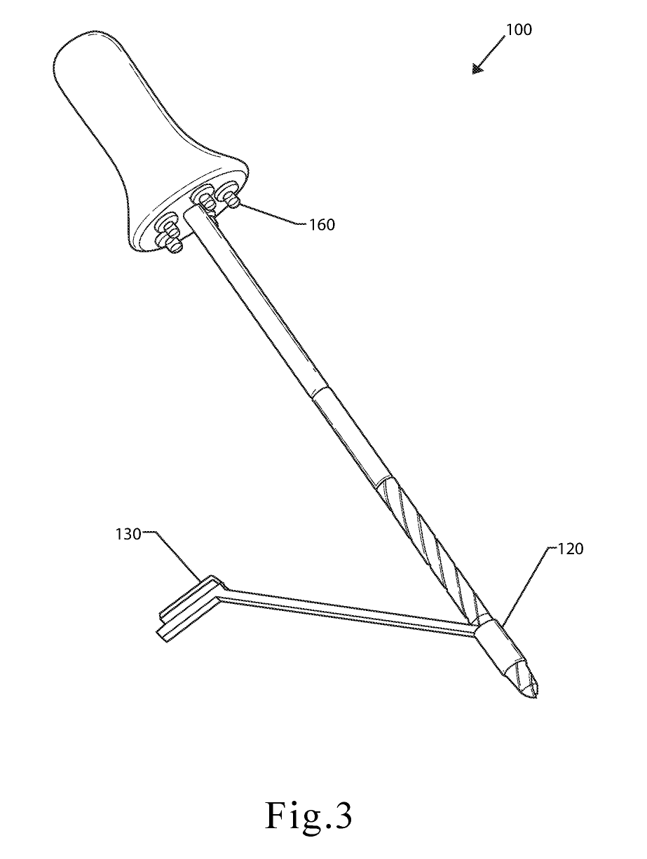

FIG. 2 and FIG. 3 are illustrations of one embodiment of a surgical instrument that is a surgical awl according to the present disclosure. As shown in FIGS. 1 and 2, the surgical awl 100 may comprise a handle 105, neck 110, drill 115, guide 120, sensor mounting portion 125, and absolute orientation sensor 130.

The handle 105 may be connected to a first end of the neck 110, and the drill 115 may be connected to a second and of the neck 110. The guide 120 may substantially encapsulate the drill 115, as shown, or alternatively the guide 120 may encapsulate the neck 110. In one embodiment, the sensor mounting portion 125 may be connected on a first end to the guide 120 and on a second end to the absolute orientation sensor 130. In the alternative embodiments, the absolute orientation sensor 130 may be mounted on substantially any portion of the surgical awl 100. The absolute orientation sensor 130 may be mounted to the surgical awl 100 by the use of an adhesive, screws, bolts, or any other connecting means. Alternatively, the absolute orientation sensor 130 may be built into the housing of the surgical awl 100.

The absolute orientation sensor 130 may comprise an accelerometer 135, gyroscope 140, magnetometer 145, device processor 150, and a power source 155.

The device processor 150 may control the absolute orientation sensor's 130 components.

The surgical awl 100 described herein may function substantially similarly to a standard medical awl, except that the surgical awl 100 provides additional orientation data to the doctor and illumination.

The orientation information generated by the absolute orientation sensor 130 may be relayed to a doctor by transmitting the orientation information to a digital display attached to a surgical instrument or to an external receiving electronic device. If the orientation information is being transmitted to an external receiving electronic device, the absolute orientation sensor 130 may comprise a wireless communication component to transmit the orientation information.

As shown in FIG. 3, the surgical awl 100 may also comprise a light source 160. The light source may be located on a proximal end of the handle 105, such that the light source 160 illuminates a portion of a patient to be operated upon. The light source 160 may interface wirelessly or wired, or both, with the surgical awl 100 or components thereof to control its operation. The light source 160 may be powered by any suitable means, including, but not limited to, connection to the power source of the surgical instrument, battery, rechargeable battery, connection to a separate power source, and the like. The light source 160 may also be a plurality of light sources, and may be located near to one another or spaced apart. In one embodiment, the light sources may be directed towards a common location such that the combination of light sources provides more lighting than a single light source would.

The surgical awl 100 may have a power source in the handle 105, or in another suitable location in order to power the light source 160 and the absolute orientation sensor 130.

The surgical awl 100 may also comprise a remote device 180, input/output device 185, communication link 190, and audio device 195. As described hereinbelow, the input/output device 185 may transmit information generated by the absolute orientation sensor 130 and processed by the device processor 150 to the remote device 180. The input/output device 185 may utilize the communication link 190 to transmit information wirelessly to the remote device 180. Additionally, the input/output device may transmit a signal to the audio device 195 to cause the audio device to provide an audio notification based on the orientation information generated by the absolute orientation sensor.

In alternative embodiments, the surgical instrument may be any instrument suitable for surgical or medical procedures or treatments on humans or animals. The surgical instrument may be made of titanium, aluminum, stainless steel, various alloys, as well as various plastics. The surgical instrument may include a housing or other outer structure to which the lighting source may be attached or affixed. In an example embodiment, the light source 160, may include a trigger mechanism or component which operates in conjunction with the operation of the surgical instrument. This may allow the light source 160 to emit light upon operation of the surgical instrument, synchronizing the operation of the light source 160 with the function of the surgical instrument. The light source 160 may include compact fluorescence lights, light emitting diodes, incandescent, fluorescent, halogen, and other types of lights.

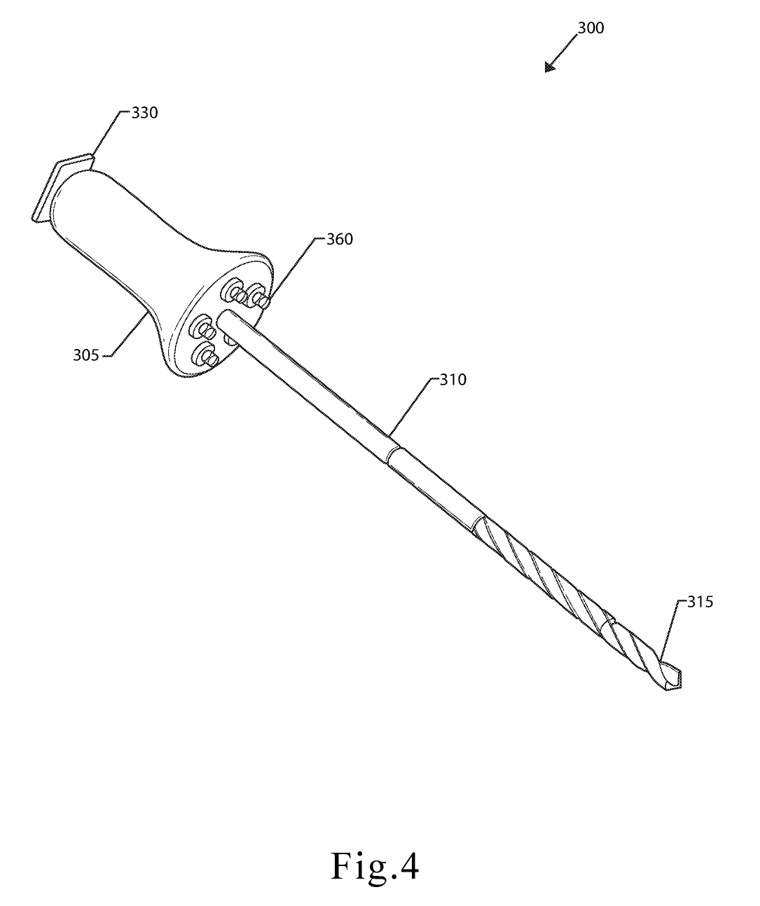

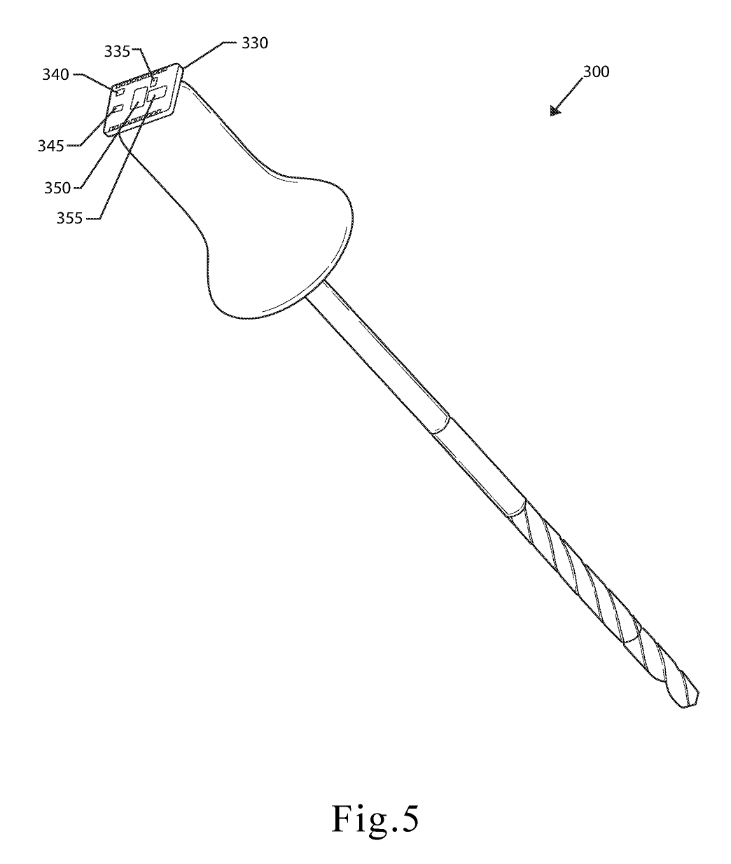

FIG. 4 and FIG. 5 are illustrations of an alternative embodiment of a surgical instrument that is a surgical awl according to the present disclosure. As shown in FIGS. 4 and 5, an alternative embodiment of a surgical awl 300 may comprise a handle 305, neck 310, drill 315, light source 360, and absolute orientation sensor 330.

The handle 305 may be connected to a first end of the neck 310, and the drill 315 may be connected to a second and of the neck 310. The absolute orientation sensor 330 may be located on a distal end of the handle 305.

As shown in FIG. 4, the surgical awl 300 made comprise a light source 360. The light source 360 may be located on a proximal end of the handle 305, such that the light source 360 illuminates a portion of a patient to be operated upon.

The surgical awl 300 may have a power source in the handle 305, or in another suitable location in order to power the light source 360 and the absolute orientation sensor 330.

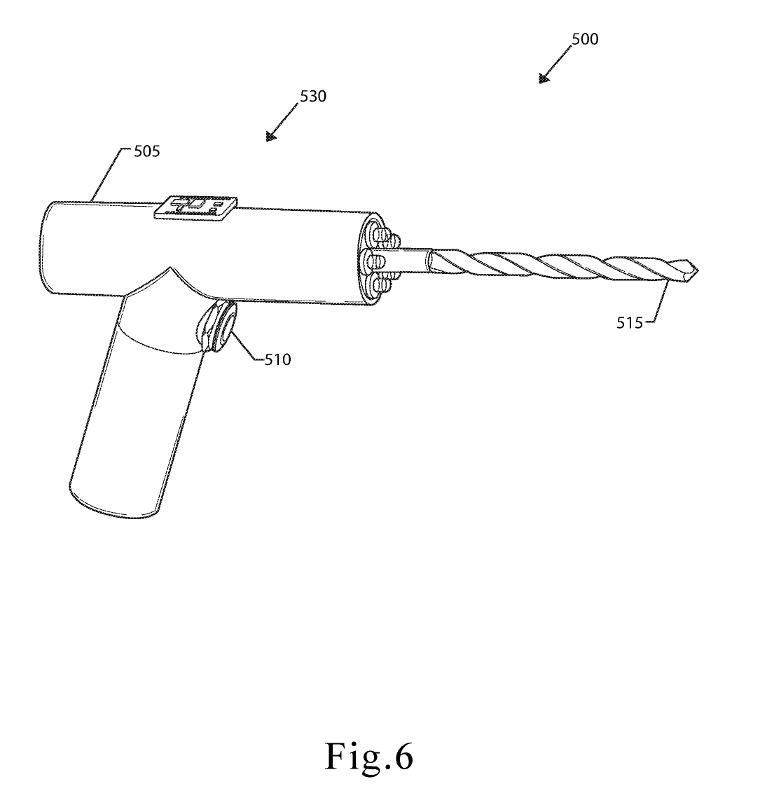

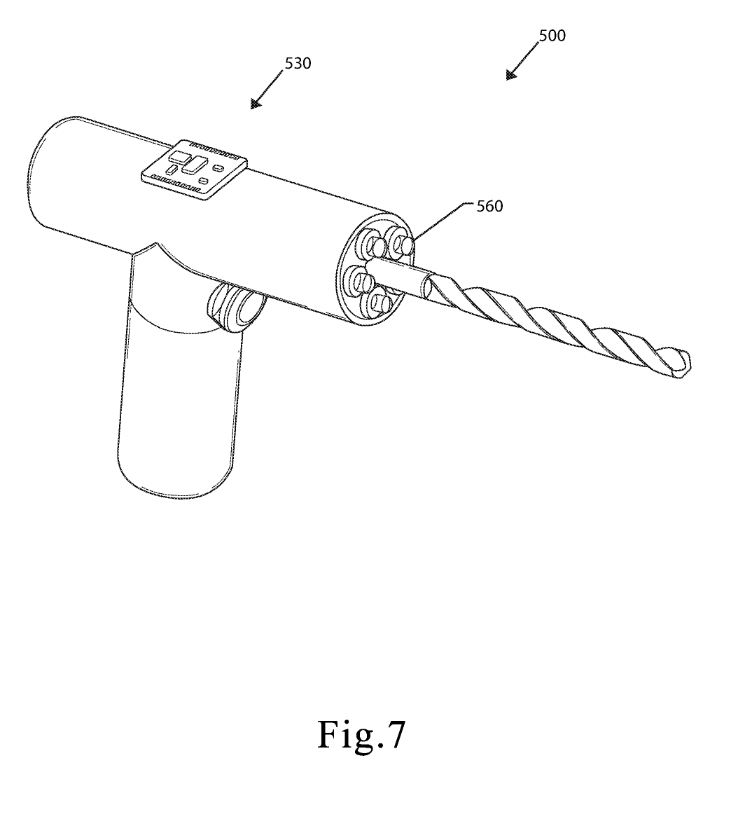

FIG. 6 and FIG. 7 are illustrations of an embodiment of a surgical instrument that is a surgical drill according to the present disclosure. The surgical drill 500 may comprise, a housing 505, trigger 510, drill 515, absolute orientation sensor 530, and light source 560. The trigger 510 may be mounted on a handle portion of the housing 505, and the drill 515 may extend from a forward portion of the housing 505. The light source 560 may be mounted near where the drill 515 and housing 505 connect. As shown in FIGS. 6 and 7, the absolute orientation sensor 530 may be located on top of the housing 505. In an alternative embodiment, the absolute orientation sensor 530 may be located on a different part of the housing 505. The absolute orientation sensor 530 may function substantially similarly to the one described in FIGS. 2-3.

A power source may be contained within the housing 505, such that the power source is able to power the drill 515, the light source 560, and/or the absolute orientation sensor 530. In one embodiment, when the trigger 510 is depressed the drill 515 and the light source 560 are activated substantially simultaneously. In a preferred embodiment, the absolute orientation sensor 530 is active independent of the trigger 510.

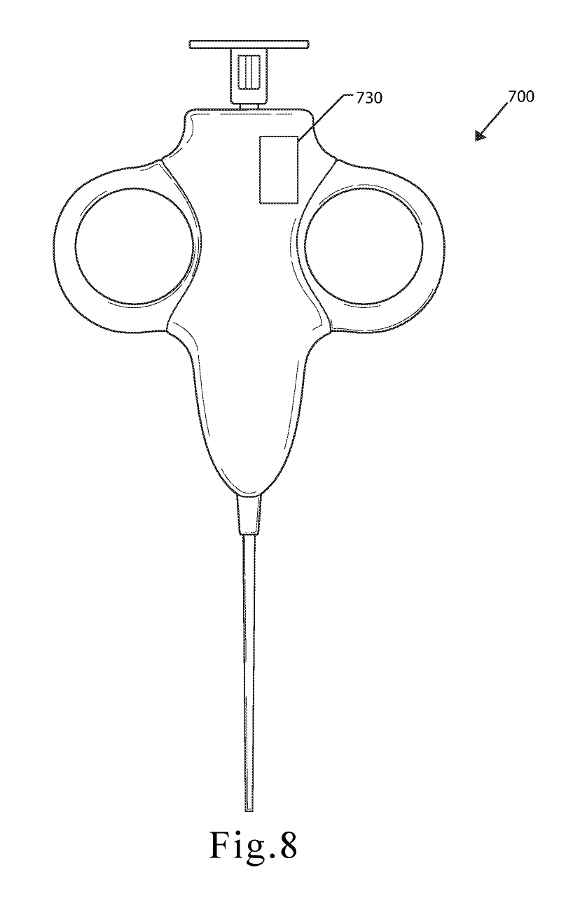

FIG. 8 is an illustration of one embodiment of a surgical instrument that is a biopsy needle according to the present disclosure. As shown in FIG. 8, the biopsy needle may comprise an absolute orientation sensor 730. The biopsy needle 700 generally comprises a hollow needle that may be inserted into an area from which a sample is desired. The biopsy needle 700 is useful in obtaining samples of certain tissues, including liver, kidney, and the genitourinary tract, skin, breast, lung, bone, prostate, testicle, intestine, brain, ovary, uterus, and thyroid, in addition to other tissue types, as well as fluid collections, abscesses, tumor, hematoma, and other masses, fluids, collections, and area of interest for potential biopsy. The biopsy needle 700 may have additional gripping portions to allow a user to more accurately and steadily use the needle to recover a sample of soft tissue. In some embodiments, the biopsy needle may be hollow, and after inserted such that a desired sample is now present in the hollow needle, a blight suction may be applied to keep the core sample inside the needle wild and needle is removed from soft tissue.

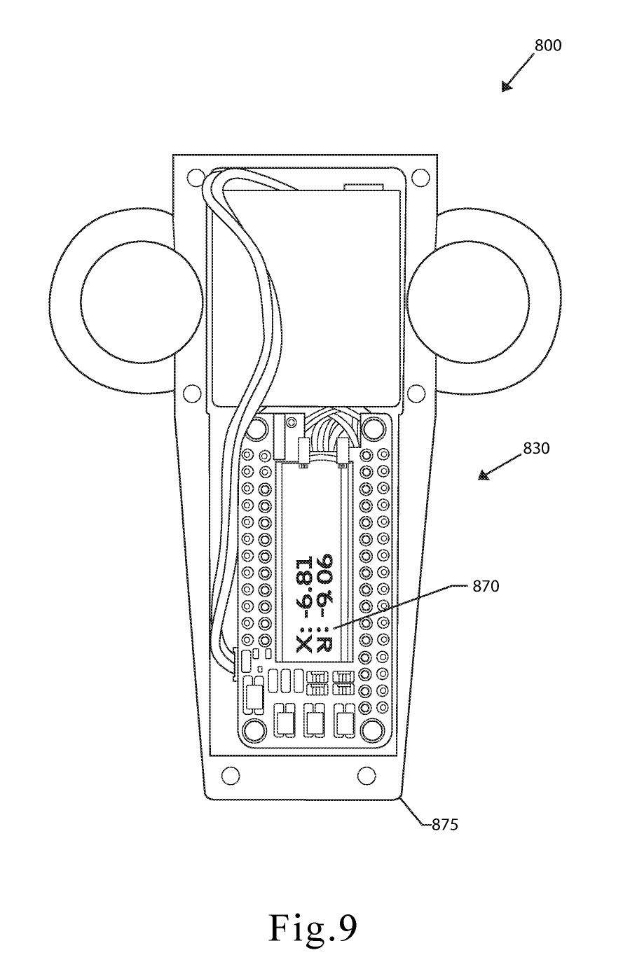

FIG. 9 is an illustration of one embodiment of an absolute orientation sensor and display. As shown in FIG. 9, the absolute orientation sensor and display 800 may comprise an absolute orientation sensor 830, display 870, and electronic housing 875. The electronic housing 875 may encapsulate the absolute orientation sensor 830 and the display 870. The electronic housing 875 may be configured to protect the absolute orientation sensor 830 and the display 875. In one embodiment, the electronic housing 875 may include a transparent portion to allow a user to view the display 870. Alternatively, the display 870 may be mounted on the outside of the electronic housing 875. The electronic housing 875 may be different shapes to accommodate different surgical tools. For example, as shown in FIG. 9, the electronic housing 875 may form of a shape that is consistent with this shape of a biopsy needle. It is understood that the electronic housing 875 that may take various shapes and make up portions of surgical instruments.

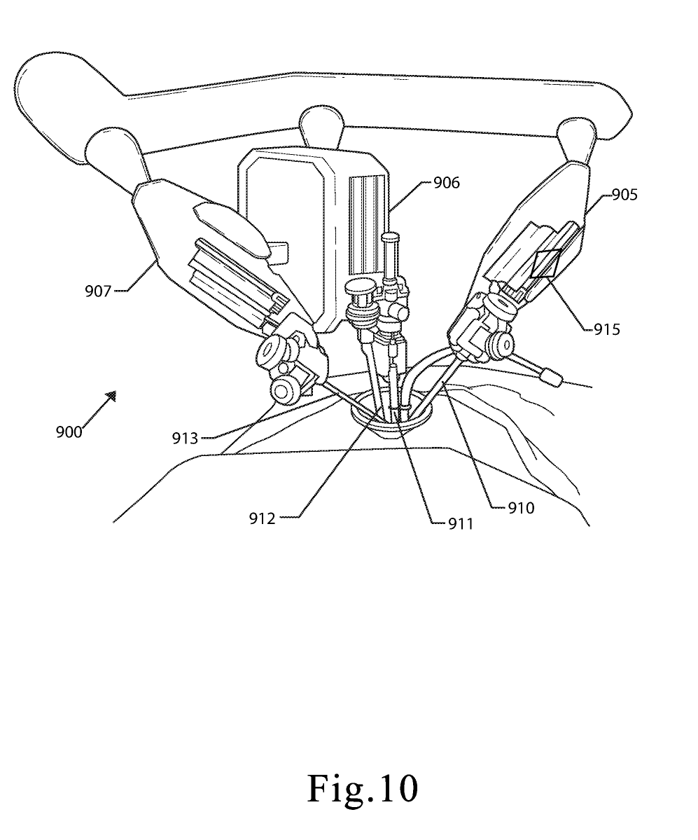

FIG. 10 is an illustration of one embodiment of a surgical instrument that is a robotic arm according to the present disclosure. As shown in FIG. 10, the robotic arm 900 may comprise mounting arms 905, 906, 907 robotic tools 910, 911, 912, 913 and an absolute orientation sensor 915. The robotic arm 900 may comprise any number of mounting arms 905, 906, 907 suitable for the surgery to be performed. Each of the mounting arms 905, 906, 907 may comprise any number of robotic tools 910, 911, 912, 913 suitable for the surgery to be performed. The robotic tools 910, 911, 912, 913 may be substantially any surgical device.

The absolute orientation sensor 915 may be substantially similar to the absolute orientation sensor 800 described in FIG. 9, and may or may not include a digital display. The absolute orientation sensor 915 may be mounted on the mounting arm 905. Alternatively, the absolute orientation sensor may be located on or in any of the mounting arms 905, 906, 907 and/or robotic tools 910, 911, 912, 913. Additionally, there may be multiple absolute orientation sensors 915 on different components of the robotic arm 900 at the same time.

In an embodiment, the surgical instrument may also comprise an input/output device coupled to a device processor, absolute orientation sensor, and/or any other electronic component of the surgical instrument. Input may be received from the device processor or absolute orientation sensor and/or output may be provided to an electronic display or another device via the input/output device. The input/output device may comprise any combinations of input and/or output devices such as buttons, knobs, keyboards, touchscreens, displays, light-emitting elements, a speaker, and/or the like. In an embodiment, the input/output device may comprise an interface port such as a wired interface, for example a serial port, a Universal Serial Bus (USB) port, an Ethernet port, or other suitable wired connection. The input/output device may comprise a wireless interface (not shown), for example a transceiver using any suitable wireless protocol, for example Wi-Fi (IEEE 802.11), Bluetooth®, infrared, or other wireless standard. For example, the input/output device may communicate with a remote device via Bluetooth® such that the data generated by the absolute orientation sensor may be sent to the remote device and monitored. In an embodiment, the input/output device may comprise a user interface.

The user interface may comprise at least one of lighted signal lights, gauges, boxes, forms, check marks, avatars, visual images, graphic designs, lists, active calibrations or calculations, 2D interactive fractal designs, 3D fractal designs, 2D and/or 3D representations of vapor devices and other interface system functions.

In an embodiment, the input/output device may comprise a touchscreen interface and/or a biometric interface. For example, the input/output device may include controls that allow the user to interact with and input information and commands to the surgical instrument. For example, with respect to the embodiments described herein, the input/output device may comprise a touch screen display. User inputs to the touch screen display are processed by, for example, the input/output device and/or the device processor. The input/output device may also be configured to process new content and communications to the surgical instrument. The touch screen display may provide controls and menu selections, and process commands and requests. Application and content objects may be provided by the touch screen display. The input/output device and/or the device processor may receive and interpret commands and other inputs, interface with the other components of the surgical instrument as required. In an embodiment, the touch screen display may enable a user to view at least one orientation data of the surgical instrument generated by the absolute orientation sensor.

In an embodiment, the input/output device may comprise an audio user interface. A speaker may be configured to send audio signals and relay the audio signals to the input/output device. The audio user interface may be any interface that is responsive to data generated. The audio user interface may be configured to transmit a sound based on the data generated by the absolute orientation sensor. The audio user interface may be deployed directly on the surgical instrument and/or via other electronic devices (e.g., electronic communication devices, such as a smartphone, a smart watch, a tablet, a laptop, a dedicated audio user interface device, other personal computing devices, and the like). The audio user interface may be used to convey data generated by the absolute orientations sensor. Such conveyance may comprise, but is not limited to, orientation data of the surgical instrument. The user may then adjust the positioning of the surgical instrument based on the audio signal output

The input/output device may be configured to interface with other remote devices, for example, computing equipment, communications devices and/or other surgical devices, for example, via a physical or wireless connection. The input/output device may thus exchange data with the other equipment. A user may sync their surgical equipment to other devices, via programming attributes such as mutual dynamic link library (DLL) ‘hooks’. This enables a smooth exchange of data between devices, as may a web interface between devices. The input/output device may be used to upload one or more orientation data to the other devices. Using monitoring equipment as an example, the one or more orientation data may comprise data such as location data, pitch data, roll data, yaw data, x, y, and z coordinate values, angular velocity data, acceleration data, magnetic field strength data, linear acceleration data, gravity data, and temperature data. Data from usage of previous monitoring sessions may be archived and shared.

In general, the embodiments herein provide surgical and medical instruments having high intensity lighting and/or absolute orientation sensors. The instruments of the present disclosure may be used in any suitable procedure or treatment which would benefit from high intensity lighting of the area to be treated or of interest, as well as knowing the orientation of such instrument in three-dimensional space. While reference is made herein to surgical instruments in particular, it should be understood that this disclosure is directed to medical, dental, or other instruments used in the treatment of humans or animals requiring lighting and/or absolute orientation.

As discussed above, the surgical instrument is any instrument suitable for surgical or medical procedures or treatments on humans or animals. The surgical instrument may be comprised of titanium, aluminum, stainless steel, various alloys, as well as various plastics. The surgical instrument may include a housing or other outer structure to which the absolute orientation sensor may be attached or affixed. In an example embodiment, the orientation sensor can include a trigger mechanism or component which operates in conjunction with the operation of the instrument. In one embodiment, the surgical instrument may be made by additive manufacturing and comprise polylactic acid (“PLA”).

An alternative embodiment of the surgical instrument may comprise Global Positioning Satellite receiving device, such that the surgical instrument may be able to determine its location on Earth.

As discussed above, any suitable surgical instrument can include the high intensity lighting source or the absolute orientation sensor, or both. Examples include, but are not limited to, powered instruments, such drills/drivers, saws, wire insertion devices, burr, and the like, and manual instruments, such as awls for pedicle screw placement, scalpels, suction, retraction devices, mallets, unpowered drills, drivers, saws, wire inserters, and burrs, and various hand tools. Surgical instruments including such high intensity lighting source and/or absolute orientation sensor can be used in any suitable procedure, treatment, or field. Examples, include, but are not limited to: orthopaedic surgery, including but not limited to: spine surgery, orthopaedic trauma surgery, foot and ankle surgery, sports surgery, joint replacement in knee, hip, shoulder, any fracture fixation, tibial, femoral, and other long bone nailing procedures, upper extremity and hand surgery, oncological orthopaedic surgery, and pediatric orthopaedic surgery; neurological surgery, including but not limited to: spine surgery, intracranial surgery of all kinds, and surgery in central and peripheral nervous systems; otolaryngology surgery; plastic surgery, obstetric and gynecological surgery, urological surgery, trauma surgery, general surgery; anaesthesia; emergency medicine; family and internal medicine; gastroenterology; cardiology; dental surgery; veterinary surgery; and all surgical and medical fields not named above.

Having thus described certain embodiments for practicing aspects of the present disclosure, it is to be appreciated that various alterations, modifications, and improvements will readily occur to those skilled in the art. Such alterations, modifications, and improvements are intended to be part of this disclosure, and are intended to be within the spirit and scope of this disclosure.

What is claimed is:

1) A surgical instrument comprising:
a device processor operable for controlling one or more components of the surgical instrument;
an absolute orientation sensing component operatively coupled to the device processor and controlled in part by the device processor, wherein the absolute orientation sensing component comprises an accelerometer, gyroscope, and magnetometer, wherein the absolute orientation sensing component is operable to detect a plurality of orientation data associated with at least one orientation condition of the surgical instrument relative to the Earth's magnetic field without requiring calibration by a separate arbitrary reference plane, and generate a plurality of orientation status data on at least a portion of the plurality of detected orientation data; and
at least one power source operatively coupled to the absolute orientation sensing component, wherein the at least one power source is operable to generate a supply of power for operation of the device processor and the absolute orientation sensing component.

2) The surgical instrument of claim 1, further comprising a display operatively coupled to the device processor, wherein the display is operable to display at least a portion of the plurality of generated orientation status data thereon.

3) The surgical instrument of claim 1, wherein the plurality of orientation data comprises at least one from the group consisting of a location data, pitch data, roll data, yaw data, linear and angular acceleration and velocity data, and direction and strength of surrounding magnetic fields data.

4) The surgical instrument of claim 3, wherein the location data comprises x, y, and z coordinate values.

5) The surgical instrument of claim 4, wherein the plurality of orientation data further comprises at least one selected from the group consisting of angular velocity data, acceleration data, magnetic field strength data, linear acceleration data, gravity data, and temperature data.

6) The surgical instrument of claim 1, further comprising an input/output device operably coupled to the device processor and controlled in part by the device processor; wherein the input/out device is operable to transmit at least a portion of the plurality of generated orientation status data to a remote device.

7) The surgical instrument of claim 1, further comprising an audio component operatively coupled to the device processor and controlled in part by the device processor, wherein the device processor is further operable to generate at least one audio response based in response to at least a portion of the plurality of generated orientation status data for output by the audio component.

8) The surgical instrument of claim 1, further comprising at least one light source operatively coupled to the device processor and controlled in part by the device processor, wherein the at least one light source is located on the surgical instrument such that the at least one light source is operable to direct light towards a specified area on an associated patient.

9) The surgical instrument of claim 8, wherein the at least one light source is a high lumen light source.

10) The surgical instrument of claim 9, wherein the at least one light source is a light emitting diode, compact fluorescent, incandescent, fluorescent, or halogen bulb.

11) The surgical instrument of claim 8, wherein the device processor is operable to activate the at least one light source when the surgical device is in use.

12) The surgical instrument of claim 1, wherein the surgical instrument is selected from the group consisting of drills, drivers, saws, wire insertion devices, burrs, awls, scalpels, suction, retraction devices, mallets, biopsy needles, unpowered tools, unpowered drills, unpowered drivers, unpowered saws, unpowered wire inserters, unpowered burrs, cutting guides, robotic arms, trocars, screw drivers, forceps, scissors, and instruments for cutting, scraping, retracting, securing, extracting, sampling, or manipulating tissue operated manually, in a powered, and robotic fashion.

13) A surgical instrument comprising:
a device processor operable for controlling one or more components of the surgical instrument;
an absolute orientation sensing component operatively coupled to the device processor and controlled in part by the device processor, wherein the absolute orientation sensing component comprises an accelerometer, gyroscope, and magnetometer, wherein the absolute orientation sensing component is operable to detect a plurality of orientation data associated with at least one orientation condition of the surgical instrument relative to the Earth's magnetic field without requiring calibration by a separate arbitrary reference plane, and generate a plurality of orientation status data on at least a portion of the plurality of detected orientation data;
at least one power source operatively coupled to the absolute orientation sensing component, wherein the at least one power source is operable to generate a supply of power for operation of the device processor and the absolute orientation sensing component; and
a visual and audio component operatively coupled to the device processor and controlled in part by the device processor, wherein the device processor is further operable to generate at least one audio response based in response to at least a portion of the plurality of generated orientation status data for output by the audio component;
wherein the plurality of orientation data comprises at least one from the group consisting of a location data, pitch data, roll data, and yaw data, angular velocity data, acceleration data, magnetic field strength data, linear acceleration data, gravity data, and temperature data; and
wherein the location data comprises x, y, and z coordinate values.

14) The surgical instrument of claim 13, further comprising an input/output device operably coupled to the device processor and controlled in part by the device processor; wherein the input/out device is operable to transmit at least a portion of the plurality of generated orientation status data to a remote device.

15) The surgical instrument of claim 13, further comprising a display operatively coupled to the device processor, wherein the display is operable to display at least a portion of the plurality of generated orientation status data thereon.

16) The surgical instrument of claim 13, further comprising at least one light source operatively coupled to the device processor and controlled in part by the device processor, wherein the at least one light source is located on the surgical instrument such that the at least one light source is operable to direct light towards a specified area on an associated patient; wherein the light source is a high lumen light source; wherein the light source is a light emitting diode, compact fluorescent, incandescent, fluorescent, or halogen bulb; wherein the device processor is operable to activate the light source when the surgical device is in use.

17) A surgical instrument comprising:
a device processor operable for controlling one or more components of the surgical instrument;
at least one light source operatively coupled to the device processor and controlled in part by the device processor, wherein the at least one light source is located on the surgical instrument such that the at least one light source is operable to direct light towards a specified area on an associated patient; wherein the light source is a high lumen light source; wherein the light source is a light emitting diode, compact fluorescent, incandescent, fluorescent, or halogen bulb; wherein the device processor is operable to activate the light source when the surgical device is in use; and
at least one power source operatively coupled to the light source, wherein the at least one power source is operable to generate a supply of power for operation of the device processor and the light source.

18) The surgical instrument of claim 17, further comprising an absolute orientation sensing component operatively coupled to the device processor and controlled in part by the device processor, wherein the absolute orientation sensing component comprises an accelerometer, gyroscope, and magnetometer, wherein the absolute orientation sensing component is operable to detect a plurality of orientation data associated with at least one orientation condition of the surgical instrument relative to the Earth's magnetic field without requiring calibration by a separate arbitrary reference plane, and generate a plurality of orientation status data on at least a portion of the plurality of detected orientation data; and
further comprising an audio component operatively coupled to the device processor and controlled in part by the device processor, wherein the device processor is further operable to generate at least one audio response based in response to at least a portion of the plurality of generated orientation status data for output by the audio component;
wherein the plurality of orientation data comprises at least one from the group consisting of a location data, pitch data, roll data, and yaw data, angular acceleration and velocity data, linear acceleration and velocity data, magnetic field strength data, gravity data, and temperature data; and wherein the location data comprises x, y, and z coordinate values.

19) The surgical instrument of claim 18, further comprising an input/output device operably coupled to the device processor and controlled in part by the device processor; wherein the input/out device is operable to transmit at least a portion of the plurality of generated orientation status data to a remote device.

20) The surgical instrument of claim 19, further comprising a display operatively coupled to the device processor, wherein the display is operable to display at least a portion of the plurality of generated orientation status data thereon.
